(19) United States
(12) Patent Application Publication
(10) Pub. No.: US 2025/0121204 A1
(43) Pub. Date: Apr. 17, 2025

(54) PORTABLE PULSED ELECTROMAGNETIC FIELD GENERATOR

(71) Applicant: Mark L. Fox, Cocoa Beach, FL (US)

(72) Inventor: Mark L. Fox, Cocoa Beach, FL (US)

(21) Appl. No.: 18/656,212

(22) Filed: May 6, 2024

Related U.S. Application Data
(60) Provisional application No. 63/590,936, filed on Oct. 17, 2023.

(51) Int. Cl. A61N 2/02

(52) U.S. Cl. CPC A61N 2/02

(57) ABSTRACT

The invention is a portable micromagnetic field generator device comprising a battery, a circuit board with a microcontroller, a memory and a current amplifier. The memory functions to store information, such as audio information, for the generation of specific pulsed magnetic field frequencies of pairs of such frequencies. The MCU uses the stored information to form a variable microcurrent, which is amplified via a current amplifier. The amplified current is used by a coil assembly to generate a variable micromagnetic field. The device is kept in close proximity to a user's body for the micromagnetic field to impart its therapeutic effects. The device also includes a display device and several buttons so that users may select the type of micromagnetic field they wish to use, as well as stop, start and pause the micromagnetic field generation.

### FIELD OF THE INVENTION

The present invention relates generally to micromagnetic field generator devices for health and wellness.

### GENERAL BACKGROUND

The use of magnetic fields in healthcare dates back several hundred years. Many people use magnetic fields, specifically pulsed electromagnetic fields (PEMF), for the treatment of acute ailments as well as for the maintenance of general health and wellness. For example, PEMF has been shown to improve the internal microcirculation and promote blood circulation, thereby enhancing the natural healing processes of the body. Magnetic fields such as PEMF may also be used in the treatment of PTSD, inflammation, arthritis, allergies, anxiety and many other illnesses. Most PEMF generators on the market are large, stationary devices that are not portable. Thus, a need exists for a portable device that can generate PEMF for the treatment of patients when they are traveling or away from home.

### SUMMARY OF THE INVENTION

In accordance with some embodiments, the present invention is a portable micromagnetic field generator device comprising a battery, a circuit board with a microcontroller (MCU), a memory and a current amplifier. The memory functions to store information, such as audio information, for the generation of specific pulsed magnetic field frequencies of pairs of such frequencies. The MCU uses the stored information to form a variable microcurrent, which is amplified via a current amplifier. The amplified current is used by a coil assembly to generate a variable micromagnetic field. The device is kept in close proximity to a user's body for the micromagnetic field to impart its therapeutic effects. The device also includes a display device and several buttons so that users may select the type of micromagnetic field they wish to use, as well as stop, start and pause the micromagnetic field generation.

### BRIEF DESCRIPTION OF THE DRAWINGS

The foregoing and other objects, features, and advantages of the invention are apparent from the following detailed description taken in conjunction with the accompanying drawings in which like parts are given like reference numerals and, wherein:

FIG. 1 depicts an angled view of the front of a portable micromagnetic field generator in accordance with embodiments of the invention.

FIG. 2 depicts an angled view of the back of a portable micromagnetic field generator in accordance with embodiments of the invention.

FIG. 3 depicts an exploded view of a portable micromagnetic field generator in accordance with embodiments of the invention.

FIG. 4 depicts a cross-sectional side view of a portable micromagnetic field generator in accordance with embodiments of the invention.

FIG. 5 depicts an electrical connection diagram of the portable micromagnetic field generator in accordance with embodiments of the invention.

FIG. 6 depicts a circuit diagram of microcontroller unit in accordance with embodiments of the invention.

FIG. 7 depicts a circuit diagram of the memory in accordance with embodiments of the invention.

FIG. 8 depicts a circuit diagram of a current amplifier in accordance with embodiments of the invention.

FIG. 9 depicts a circuit diagram of a battery in accordance with embodiments of the invention.

FIG. 10 depicts a circuit diagram of a display device in accordance with embodiments of the invention.

FIG. 11 depicts a circuit diagram of a user input component in accordance with embodiments of the invention.

FIG. 12 depicts a circuit diagram of a charging interface in accordance with embodiments of the invention.

The images in the drawings are simplified for illustrative purposes and are not depicted to scale. Within the descriptions of the figures, similar elements are provided similar names and reference numerals as those of the previous figure(s). The specific numerals assigned to the elements are provided solely to aid in the description and are not meant to imply any limitations (structural or functional) on the invention.

The appended drawings illustrate exemplary configurations of the invention and, as such, should not be considered as limiting the scope of the invention that may admit to other equally effective configurations. It is contemplated that features of one configuration may be beneficially incorporated in other configurations without further recitation.

### DETAILED DESCRIPTION

The embodiments of the disclosure will be best understood by reference to the Figures, wherein like parts are designated by like numerals throughout. It will be readily understood that the components, as generally described and illustrated in the Figures herein, could be arranged and designed in a wide variety of different configurations or be entirely separate. Thus, the following more detailed description of the embodiments of the device of the disclosure, as represented in the Figures is not intended to limit the scope of the disclosure, as claimed, but is merely representative of possible embodiments of the disclosure.

The following description sets forth numerous embodiments and parameters. It should be recognized, however, that such description is not intended as a limitation on the scope of the present invention but is instead provided as a description of exemplary embodiments. Various modifications to the examples described will be readily apparent to those of ordinary skill in the art, and the general principles defined may be applied to other examples and applications without departing from the spirit and scope of the invention. Thus, the present invention is not intended to be limited to the examples described herein but is to be accorded a scope consistent with the claims.

Generally, the invention is a variable micromagnetic field generator, which may comprise a housing assembly, which includes a hollow cavity. A circuit board and a battery may be fixedly arranged in the housing assembly. The circuit board comprises a microcontroller unit (MCU), a memory component and a current amplifier. The memory stores information used for the generation of specific magnetic fields frequencies, pairs of magnetic fields frequencies variations or compositions of a variety of magnetic field frequencies or pairs of magnetic fields frequencies. The information may be audio information or any other information capable of producing a specific frequency. The MCU decodes the information in the memory to form a variable microcurrent and the current amplifier amplifies the variable micro current. A coil assembly, fixedly arranged in the housing assembly, is in electrical communication with the current amplifier. The coil assembly with an electrical current in accordance with the information produces a variable micro magnetic field. The device further includes a display component electrically connected with the circuit board. The display component penetrates the housing component such that the display component defines a portion of the exterior of the device. A user input component is in communication with the circuit board and penetrates through the housing component to the exterior of the device.

In some embodiments, the device generates pulsed electromagnetic fields (PEMF). PEMF generators provide a myriad of benefits over their static magnet counterparts including being cheaper, smaller and capable of generating stronger magnetic fields.

In some embodiments, the housing assembly comprises a main housing and a cover plate, which are detachably connected by any appropriate means such as a buckle, fastener, slide, etc. The detachable design of the main housing and cover plate allows for the maintenance and repair of the components in the housing assembly. The housing assembly may be any shape suitable to contain the components necessary for micromagnetic field generation, such as rectangular, square, circular or any polygonal shape.

In some embodiments, a plurality of holes are arranged through the main housing, and the holes correspond with the positions and dimensions of the display device and the user control component.

In some embodiments, a heat dissipation device is arranged in the housing assembly, and the coil assembly is fixedly arranged on the heat dissipation device allowing for the heat conduction of the coil assembly and prolonging the service life of the coil assembly. In some embodiments, the heat dissipation device comprises a metal alloy (such as an aluminum alloy) heat dissipation bracket. A heat insulation pad may be arranged between the heat dissipation device and the main shell thereby preventing the heat of the heat dissipation device from being transmitted to the main housing.

In some embodiments, a plurality of heat dissipation holes are arranged through the cover plate. The positions of the plurality of heat dissipation holes correspond to the position of the coil assembly allowing for heat conduction of the coil assembly and thereby prolonging the service life of the coil assembly.

In some embodiments, the circuit board is provided with a temperature sensor which is electrically connected with the MCU and operable to monitor the temperature of coil assembly. The temperature sensor allows the detection and quantification of abnormal conditions, especially short circuit conditions, and allows such conditions to be handled in a timely manner and without damage to the components of the device.

In some embodiments, the user input assembly comprises a plurality of buttons, wherein the buttons are arranged on the circuit board and penetrate the main housing and accessible from the outside of the housing. The user input assembly may comprise one or more knobs, switches, buttons, dials, touchpads or any other suitable control means.

In some embodiments, the battery adopts a rechargeable battery. A charging interface is arranged on the circuit board, and the charging interface penetrates the housing component such that it is convenient to charge the battery.

In some embodiments, the coil assembly adopts a double coil allowing for an increase of the stability of the micromagnetic field. In some embodiments, the coil may be circular, rectangular, square, or any polygonal shape suitable to create a magnetic field.

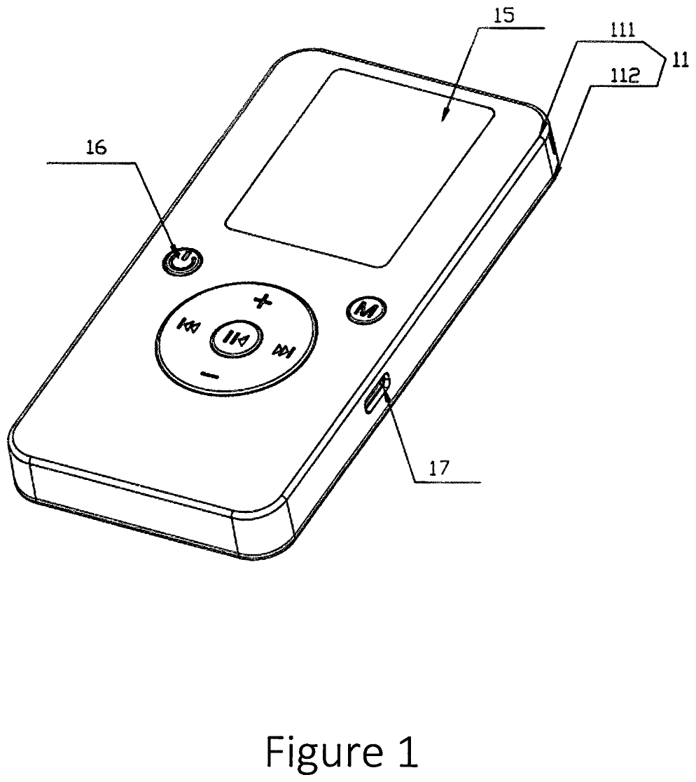

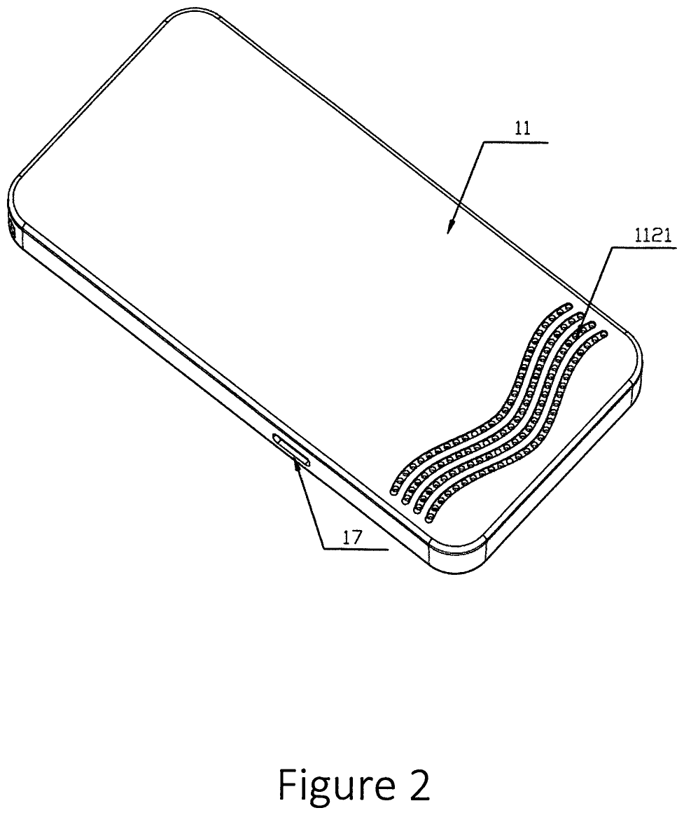

Referring to FIGS. 1 to 4, in some embodiments the variable micromagnetic field generator includes a housing assembly 11, a circuit board 12, a coil assembly 13, a battery 14, a display device 15, a user input assembly 16, a charging interface 17 and a heat dissipation device 18. The circuit board 12 and battery 14 are attached inside housing assembly 11. The circuit board 12 may be electrically connected to the coil assembly 13, battery 14, display device 15, user input assembly 16, and the charging interface 17. The battery 14 may provide power for the circuit board 12, the display device 15, user input assembly 16 and other components. The coil assembly 13 may be attached inside housing assembly 11 and in close proximity to the inner wall of the housing assembly 11 in order to reduce the number physical impediments that may obstruct effective transmission of the micromagnetic field generated by the coil assembly 13. Coil assembly 13 may generate a variable micromagnetic field through a variable current. Display device 15 may be electrically connected to the circuit board 12 and embedded in main housing 111 such that it is easily accessible to users. Display device 15 may be used for human-computer interaction and real-time display. User input assembly 16 and charging interface 17 may be arranged on circuit board 12. User input assembly 16 may control the power, function and/or settings of the micromagnetic filed generating device. Charging interface 17 may be used to charge battery 14. Heat dissipation device 18 may be arranged in housing assembly 11, and coil assembly 13 may be arranged on heat dissipation device 18. Heat dissipation device 18 may function to transmit the heat generated by the coil assembly 13.

Referring to FIGS. 1 to 4, in some embodiments housing assembly 11 includes a main housing 111 and a cover plate 112. The cover plate 112 may be connected with the main housing 111 through a buckle, snap, slide, latch, screw or any other attachment means common in the art. Cover plate 112 and main housing 111 may form a hollow cavity. Coil assembly 13, battery 14, display device 15, user input assembly 16, charging interface 17, and heat dissipation device 18 may be arranged in the hollow cavity. A plurality of through holes 1111 are arranged through the main housing 111, and the positions and dimensions of the through holes 1111 correspond to positions and dimensions of display device 15 and user input assembly 16. A plurality of heat dissipation holes 1121 are arranged through the cover 112. The positions of the plurality of heat dissipation holes 1121 correspond to the positions of coil assembly 13, so that the heat of coil assembly 13 may be transmitted from the housing assembly 11. The plurality of heat dissipation holes 1121 may further facilitate the transmission of the micromagnetic field generated by coil assembly 13.

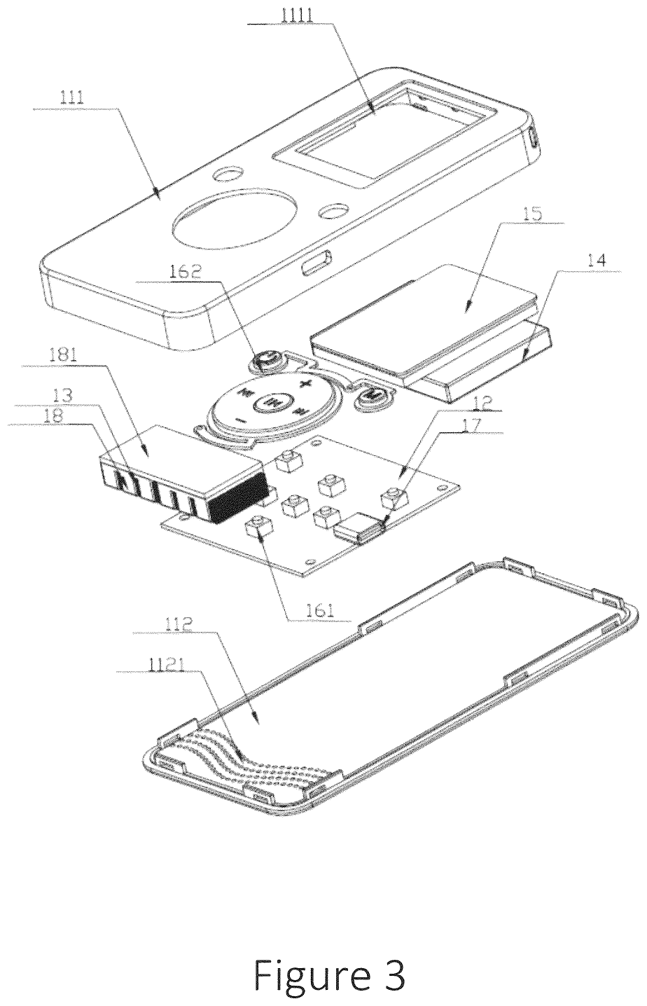

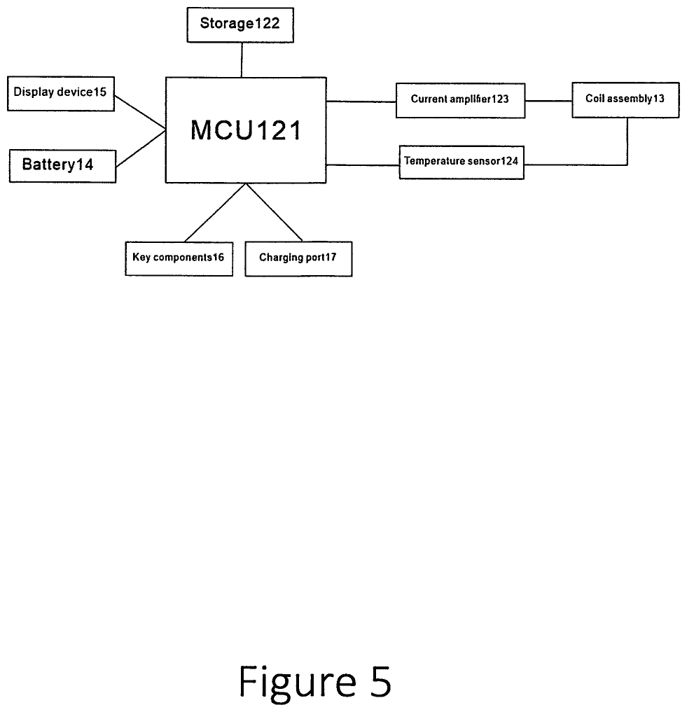

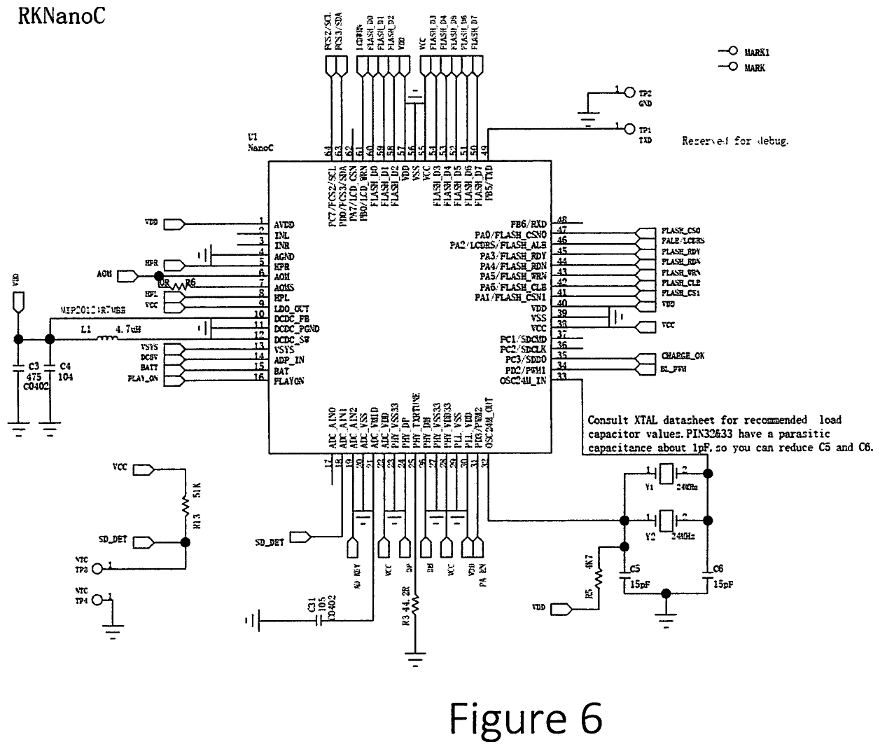

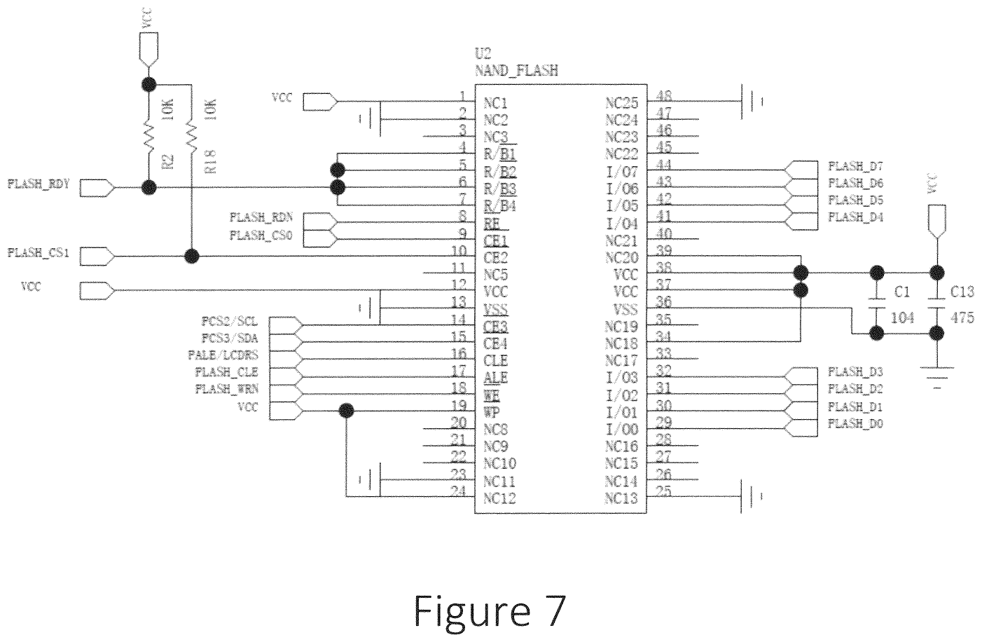

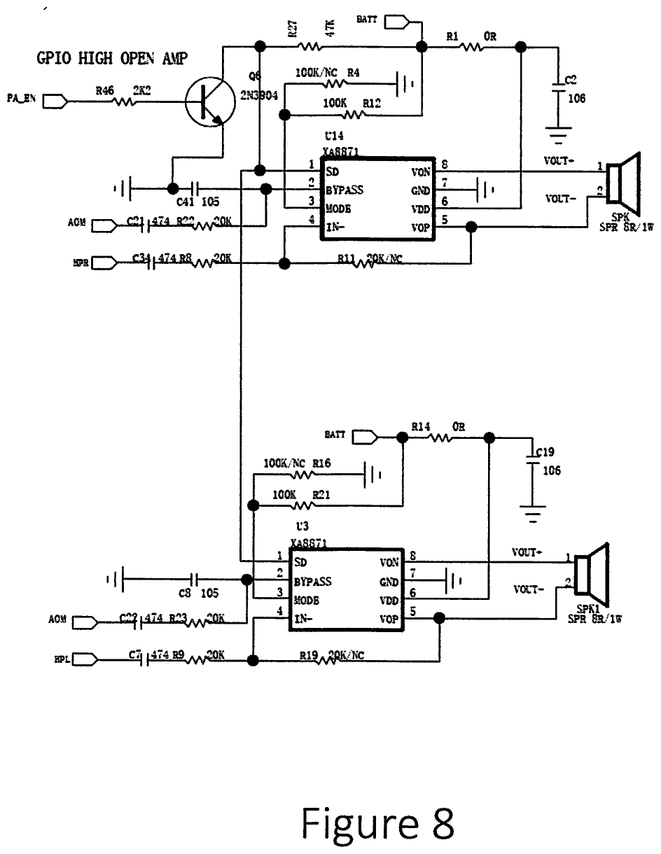

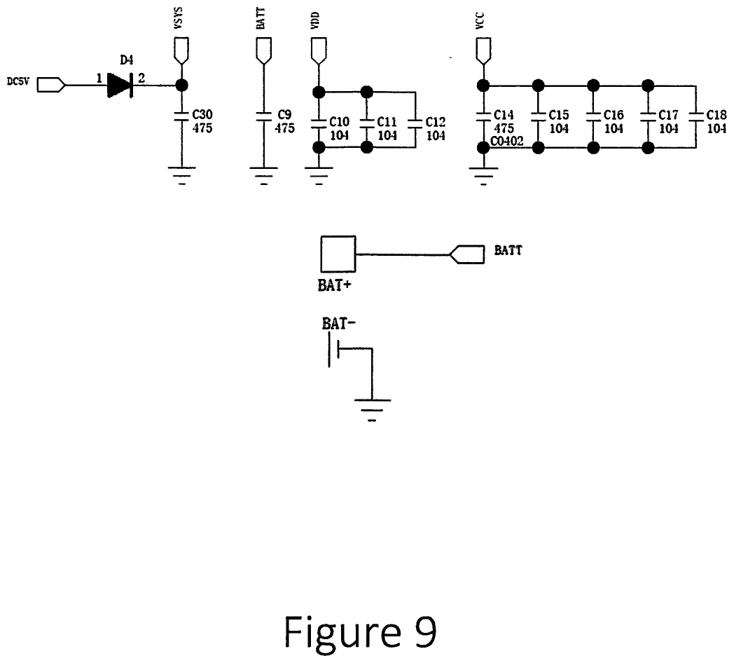

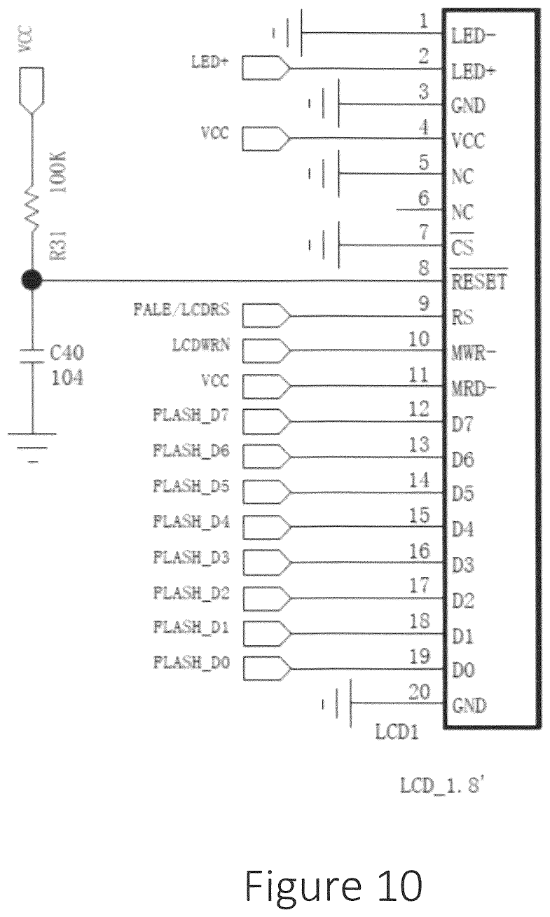

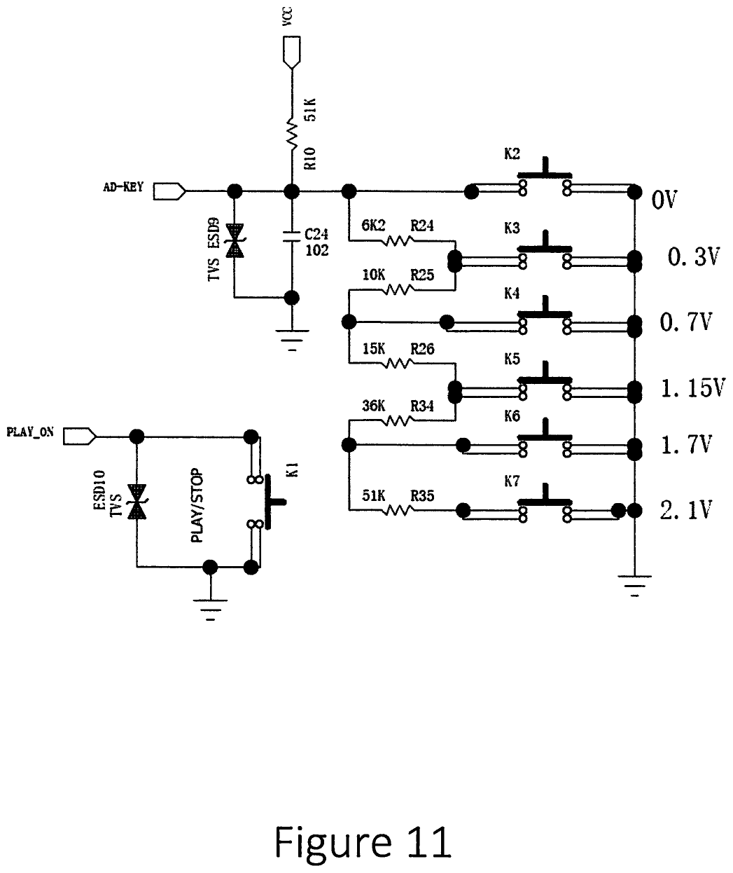

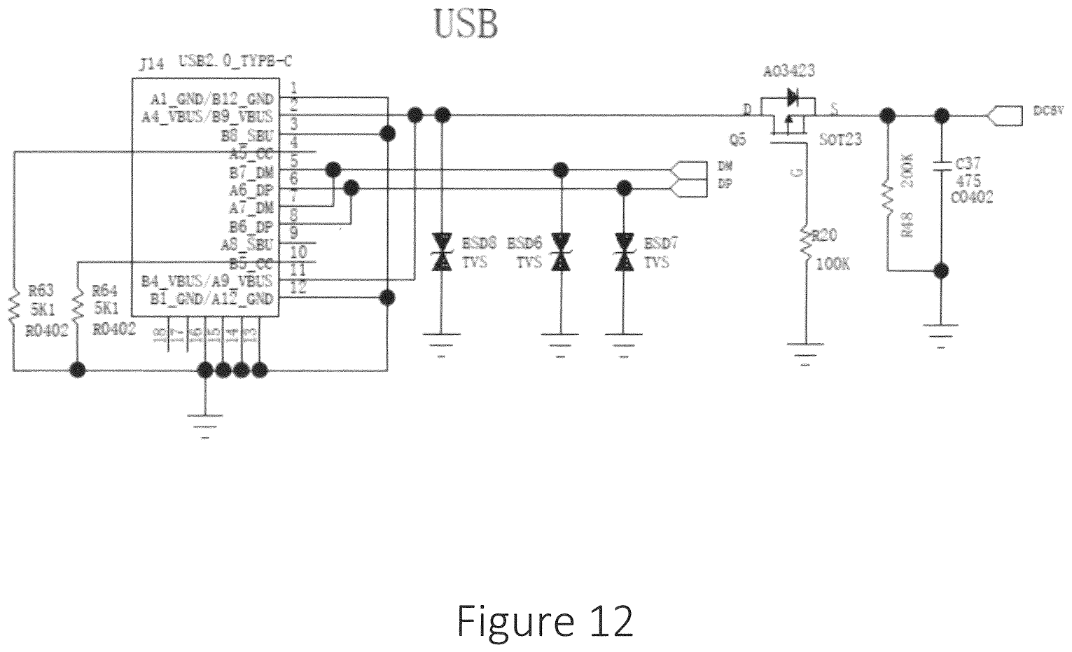

Referring to FIGS. 3 to 12, in some embodiments, the circuit board 12 includes MCU 121, memory 122 and current amplifier 123. The MCU 121 is electrically connected to memory 122, the current amplifier 123, the battery 14, the display device 15, the user input assembly 16, and the charging interface 17. Memory 122 can store a plurality of information, such as audio or digital information. In some embodiments, the information used to generate PEMFs is stored in the .mp3 format. MCU 121 can decode the information in memory 122 to form a variable micro current. Current amplifier 123 may be electrically connected with coil assembly 13, which amplifies the variable micro current of the MCU 121, transmits it to coil assembly 13, and generates a variable micromagnetic field through coil assembly 13. Circuit board 12 may be provided with a temperature sensor 124, which can be electrically connected with the MCU 121 and in contact with coil assembly 13. Temperature sensor 124 can monitor the temperature of the coil assembly 13 and transmit the temperature readings to MCU 121 in real time for processing.

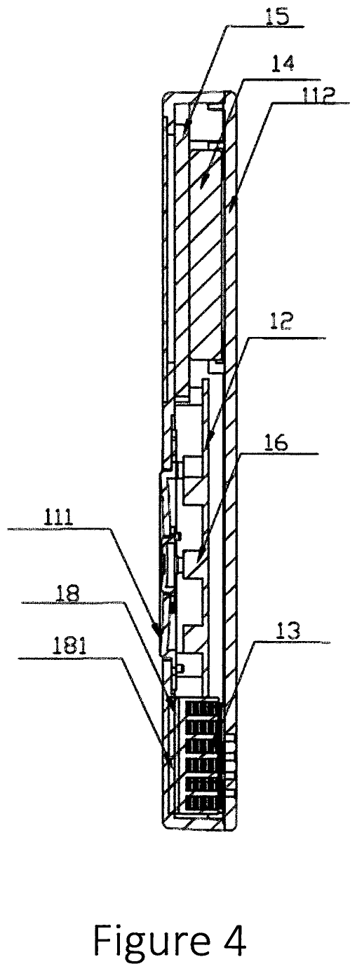

Referring to FIGS. 3 and 4, in some embodiments, the coil assembly 13 uses a double coil. A double coil may effectively enhance the generated magnetic fields by increasing its stability. Battery 14 may be a rechargeable battery, for example, battery 14 may be polymer lithium ion, nickel cadmium, lead-acid, alkaline, lead or any other rechargeable battery type that is common to in the art. Display device 15 may be any suitable display common in the art, for example, an LED display or LCD display. Display device 15 may be used to display device settings, treatment options, micromagnetic field options, data about the device's functioning, data about the magnetic field being generated or any other information relevant to the functioning of the device. In some embodiments, the display device may be a touch screen component capable of receiving inputs.

Referring to FIGS. 1, 3 and 4, in some embodiments, the user input assembly 16 includes a plurality of user input bodies 161 and user input housings 162. The plurality of user input bodies 161 are arranged on the circuit board 12 and run through the main housing 111. The user input buttons 162 are arranged on a plurality of user input bodies 161. The user input buttons 162 may be integrally formed, and use any suitable material, such as rubber. The switch and function settings of the device can be controlled by manipulating the user input bodies 161 via user input buttons 162 or other input controls. The user input assembly 16 can be used to select different information from memory in order to have the device generate different micromagnetic fields to treat different illnesses or conditions.

Charging interface 17 may be a USB interface, micro-USB interface, Lightning interface, USB-C interface or any other suitable charging interface. In the shown embodiment, the charging interface 17 adopts a USB-C interface.

Referring to FIGS. 3 and 4, in some embodiments, the heat dissipation device 18 uses a heat dissipation bracket wherein coil assembly 13 is attached to the heat dissipation bracket. A thermal insulation pad 181 may be arranged between the heat dissipation device 18 and the main housing 111. The thermal insulation pad 181 may be a silicone thermal insulation pad or any form of insulating material common in the art.

In some embodiments of the invention, the generated magnetic fields are between 0 and 900 micro tesla in strength. In some embodiments of the invention, the generated magnetic fields are between 0 and 1200 micro tesla in strength. In some embodiments of the invention, the generated magnetic fields are between 0 and 1500 micro tesla in strength.

For the purposes of promoting an understanding of the principles of the invention, reference has been made to the preferred embodiments illustrated in the drawings, and specific language has been used to describe these embodiments. However, this specific language intends no limitation of the scope of the invention, and the invention should be construed to encompass all embodiments that would normally occur to one of ordinary skill in the art. The particular implementations shown and described herein are illustrative examples of the invention and are not intended to otherwise limit the scope of the invention in any way. For the sake of brevity, conventional aspects of the system (and components of the individual operating components of the system) may not be described in detail. Furthermore, the connecting lines, or connectors shown in the various figures presented are intended to represent exemplary functional relationships and/or physical or logical couplings between the various elements. It should be noted that many alternative or additional functional relationships, physical connections or logical connections may be present in a practical device. Moreover, no item or component is essential to the practice of the invention unless the element is specifically described as “essential” or “critical”. Numerous modifications and adaptations will be readily apparent to those skilled in this art without departing from the spirit and scope of the present invention.

1. An electromagnetic field therapy device comprising:
A microcontroller;
wherein the microcontroller is in communication with a memory component;
wherein the microcontroller is in communication with a battery;
wherein the microcontroller operates to convert audio formatted files stored on said memory component to a current;
wherein said microcontroller is in communication with a coil assembly such that the current generated by said microcontroller causes the coil assembly to produce a magnetic field.

2. The device of claim 1, wherein the magnetic field is generated by a coil assembly.

3. The device of claim 1, wherein a current amplifier is in communication with the microcontroller and coil assembly.

4. The device of claim 3, wherein the current generated by said microcontroller is amplified by a current amplifier before traveling to the coil assembly.

5. The device of claim 1, wherein the magnetic field is generated is between 0 and 900 μT.

6. The device of claim 1, wherein the device further comprises a display component connected to the microcontroller.

7. The device of claim 1, wherein the audio formatted files is an MP3 file.

8. The device of claim 1, wherein the power source is a battery.

9. The device of claim 1, wherein the device is portable.

10. The device of claim 1, wherein the device comprises a heat dissipation device.
